(19) United States
(12) Patent Application Publication
(10) Pub. No.: US 2025/0000443 A1
(43) Pub. Date: Jan. 2, 2025

(54) A WEARABLE TO ASSESS KNEE JOINT INTEGRITY USING NON-CONTACT ACOUSTIC SENSORS

(71) Applicant: ECOLE POLYTECHNIQUE FEDERALE DE LAUSANNE (EPFL), Lausanne (CH)

(72) Inventors: Jérôme THEVENOT, Valeyres-Sous-Ursins (CH); Kamiar AMINIAN, La Tour-de-Peilz (CH); David ATIENZA ALONSO, Echandens-Denges (CH)

(21) Appl. No.: 18/708,959

(22) PCT Filed: Nov. 30, 2021
(86) PCT No.: PCT/IB2021/061126

§ 371 Date: May 9, 2024

(51) Int. Cl. A61B 5/00

(52) U.S. Cl. CPC A61B 5/4585; A61B 5/6828; A61B 5/6831; A61B 5/7475; A61B 2560/0443; A61B 2560/0462; A61B 2562/0204; A61B 2562/0219; A61B 2562/164

(57) ABSTRACT

A smart wearable (SW) for collecting data on an integrity of a joint of a wearer, the wearable including a first frame (1) configured to be attached in proximity of the joint of the wearer, the frame (1) including an elastic sensor frame (3) having an open cavity (21) configured to be attached to an area of interest of the joint, a kinematic sensor device (19) configured to measure kinematics of the joint, and acoustic sensor device (13) arranged in the cavity (21) configured to capture airborne acoustic emissions from the area of interest, and a data processor device (18) configured to receive and record data from the kinematic sensor device (19) and from the acoustic sensor device (13).

### FIELD OF THE INVENTION

The present invention relates to method, system, and devices for analyzing and gathering data of a joint, for example a knee joint, and devices for non-invasive joint analysis. The invention also relates to the field smart wearable devices and sensors.

### BACKGROUND

Joint pain is one of the leading causes of disability worldwide. Knees are the most affected by it, due to different injuries or disorders, for example but not limited to arthritis, anterior cruciate ligament (ACL) tears, runner's knee. See Nguyen et al., “Increasing Prevalence of Knee Pain and Symptomatic Knee Osteoarthritis: Survey and Cohort Data,” Annals of internal medicine, Vol. 155, No. 11, year 2011, pp. 725-732. The leading cause of knee pain is osteoarthritis (OA), one of the most debilitating diseases characterized by chronic discomfort, cartilage deterioration, tenderness, stiffness, crepitus (joint noise), and inflammation. With such a high impact on mobility, OA denies many people an active and independent lifestyle. As the disease is highly heterogeneous, a single therapeutic, for example targeting the joint tissue solely, is not effective. Improvement of patient stratification is required to develop better and personalized OA treatments.

Besides OA, other disorders are affecting the knee and inducing knee pain: ligament injuries (typically anterior cruciate ligament ACL) and meniscal tears. The occurrence of either of these conditions increases the risk for OA, especially if not addressed in time. An ACL injury is a tear or section of the anterior cruciate ligament that assures the joint's stability. These injuries commonly occur during sports. The condition is typically diagnosed by physical examination at the practitioner's office, by subjective assessment of the range of motion of the joint using different tests; this results in specificity and accuracy in the range of 80%. However, less than 15% of the diagnoses are made during the first visit, typically three consultations are required. Depending on the severity of the ACL injury, treatment may include rest and rehabilitation exercises. The meniscus is a piece of cartilage that provides a cushion between the femur and the tibia, acting as a “shock absorber” in the joint. Like ACL, a torn meniscus can result from any activity that involves aggressive pivoting or sudden stops and turns. The diagnosis is obtained typically during physical examination, either with a McMurray test, which puts tension on the meniscus and results in a clicking noise, or the Thessaly test to assess patient discomfort. Both tests are highly subjective with moderate accuracy (McMurray: 56-84%, Thessaly: 61-96%), suggesting the need for magnetic resonance imaging (MRI) to confirm the diagnosis and its severity. The treatment varies based on the severity. It consists of either physiotherapy, partial meniscectomy, or meniscus repair surgery.

Today, proper diagnosis of OA typically occurs only in the later stages of the disease once it has manifested itself, and clear signs can be detected through patient history, clinical symptoms, and radiographic signs. The conventional modality to evaluate the knee at advanced stages of OA is X-ray imaging which cannot show the affected soft tissues. Up to 50% of the cases show changes not necessarily associated with joint pain. See Behzad Heidari, “Knee Osteoarthritis Prevalence, Risk Factors, Pathogenesis and Features: Part I,” Caspian Journal of Internal Medicine, Vol. 2, No. 2, year 2011, p. 205. Moreover, one main drawback of radiography, and any other diagnostic imaging modalities, is that the information delivered is a static picture that does not represent the actual function of the joint: the motion. Furthermore, X-ray involves radiation and requires trained personnel and established infrastructures.

U.S. Patent No. 2021/0153804 describes a joint analysis probe having a tubular frame 102 that has an elongated shape that is dimensioned to be hand-held by a user, or attachable to a knee brace, this reference herewith incorporated by reference in its entirety. The probe also includes a microphone embedded into the frame, configured to measure sounds from a joint of a subject in a non-contact manner. However, this solution has several drawbacks with respect to the attachment options, generation of parasitic acoustic waves from the knee brace, reliability and consistency of the microphone placement, and placement accuracy.

Despite all these advancements in the field of knee joint analysis and diagnostics, and the treatment of knee disorders and diseases such as OA, in light of the several drawbacks of the existing methods and techniques enumerated above with respect to the acoustic joint probing and diagnosis, advanced and more sophisticated solutions for joint knee analysis and data gathering are strongly desired.

### SUMMARY

According to one aspect of the present invention, a smart wearable for collecting data on an integrity of a joint of a wearer is provided. Preferably, the smart wearable includes a first frame configured to be attached in proximity of the joint of the wearer, the frame including an elastic sensor frame having an open cavity configured to be attached to an area of interest of the joint, a kinematic sensor device configured to measure kinematics of the joint, and acoustic sensor device arranged in the cavity configured to capture airborne acoustic emissions from the area of interest, and a data processor device configured to receive and record data from the kinematic sensor device and from the acoustic sensor device.

According to another aspect of the present invention, the joint is a knee joint, and the smart wearable preferably includes a second frame, wherein the kinematic sensor device includes a first motion sensor and a second motion sensor, wherein the first frame is attachable to an upper shin of the wearer, the first frame including the acoustic sensor device, and the second frame is attachable to a thigh of the wearer, and wherein the first motion sensor is arranged at the first frame, and the second motion sensor is arranged at the second frame.

According to still another aspect of the present invention, preferably, the cavity is configured to be placed at a subpatellar or infrapatellar area of the knee joint, the acoustic sensor device arranged in the cavity to capture airborne acoustic emissions from the subpatellar or infrapatellar area, a sensing area of the acoustic sensor device not contacting the skin of the wearer.

According to yet another aspect of the present invention, a system for analyzing an integrity of a knee joint of a wearer is provided. Preferably, the system includes a smart wearable (SW) for collecting data on the integrity of the knee joint, the smart wearable system including a data transmission device for transmitting data recorded from the kinematic sensor device and the acoustic sensor device, and a data processing device (DP) configured to receive the data from the smart wearable (SW) and to analyze at least one of kinematics and acoustics of the knee joint.

The above and other objects, features and advantages of the present invention and the manner of realizing them will become more apparent, and the invention itself will best be understood from a study of the following description and appended claims with reference to the attached drawings showing some preferred embodiments of the invention.

### BRIEF DESCRIPTION OF THE SEVERAL VIEWS OF THE DRAWINGS

The accompanying drawings, which are incorporated herein and constitute part of this specification, illustrate the presently preferred embodiments of the invention, and together with the general description given above and the detailed description given below, serve to explain features of the invention.

FIG. 1 shows different aspects of the smart wearable SW according to some aspects of the present invention, showing a leg with a knee joint of wearer or subject, with an exemplary representation of an embodiment of the smart wearable SW arranged in proximity of the knee joint, having a first lower frame 1 and a second upper frame 2;

FIG. 2 showing different top, side and bottom views of an exemplary smart wearable SW that is made of first and second frames 1, 2;

FIGS. 3A and 3B show different aspects of the elastic sensor frame 3 that can be attached to the first frame 1, with FIG. 3A showing a top, side, and bottom view showing an elastic sensor frame 3 having a curved-shaped soft layer 12 for engagement and with a sensing area of a joint, for example the subpatellar or infrapatellar area of a knee joint, and having a cavity 21 with an acoustic sensor device 13 therein, cavity 21 having an opening in the skin facing ergonomic surface 16 facing a skin of the subpatellar or infrapatellar area of a knee joint, and FIG. 3B showing a cross-sectional view of elastic sensor frame 3 placed onto subpatellar or infrapatellar area of the knee joint;

FIG. 4 showing a schematic and exemplary view of the different functional components of the smart wearable SW as a system with an external data processing device DP, data processing device DP configured to access a memory or a database for performing signal analysis;

FIG. 5 showing different exemplary steps of a method that can be performed with the data received and stored from the smart wearable SW, for example computational steps that can be performed by the data processing device DP; and

FIG. 6 showing different curves of data that can be acquired by the smart wearable, showing an angle of the knee bending as a function of time determined from kinematic sensor device 19, 20, for example two Inertial Measurement Units (IMU), showing a first captured acoustic signal from medial subpatellar acoustic emissions captured by first microphone 13.1, and showing a second captured acoustic signal from lateral subpatellar acoustic emissions captured by second microphone 13.2.

Herein, identical reference numerals are used, where possible, to designate identical elements that are common to the figures. Also, the images in the drawings are simplified for illustration purposes and may not be depicted to scale.

### DETAILED DESCRIPTION OF THE PREFERRED EMBODIMENTS

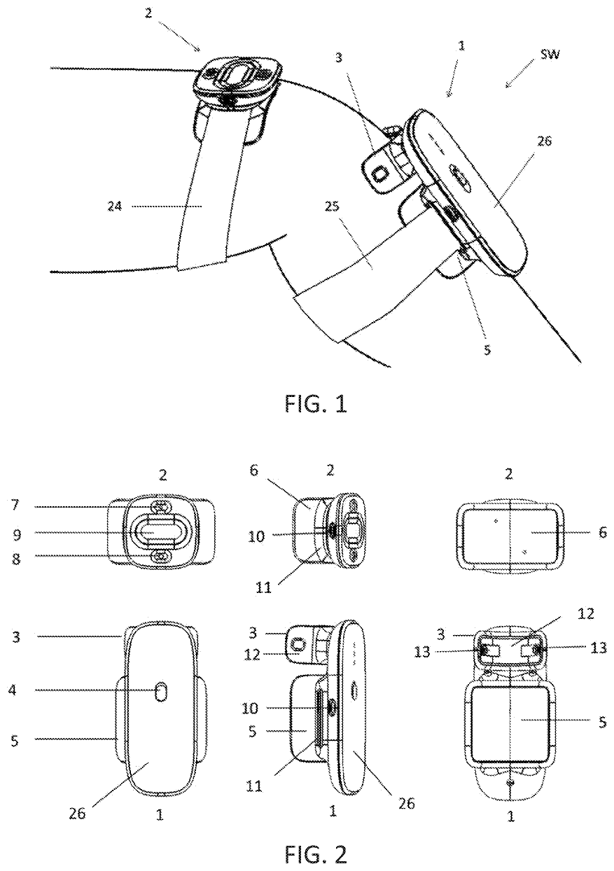

FIG. 1 shows different aspects of the smart wearable SW according to some aspects of the present invention, depicting a perspective view with a smart wearable SW having a first and second frame 1, 2 attached in close proximity to a joint of a subject, for example a human knee joint. In the embodiment presented in FIG. 1, smart wearable SW can include a first lower frame or case 1 attachable to the upper shin of the subject, for example with a releasable strap 24 as a first attachment mechanism, the lower frame 1 having an elastic sensor frame 3 attached thereto, and can optionally include a second upper frame or case 2 attachable to the thigh of the subject or wearer, with a releasable strap 25 as a first attachment mechanism. Other types of first and second attachment mechanisms 24, 25 can be used, for example but not limited to elastic bands, belts, tubular flexible sleeve such as neoprene sleeves, ribbons, Velcro™ bands, laces, or a combination of these elements. As indicated above, it is possible that the smart wearable SW includes only one element, namely the lower frame 1.

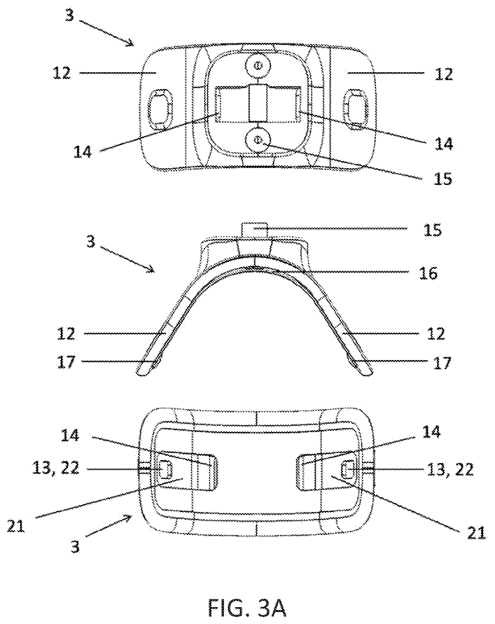

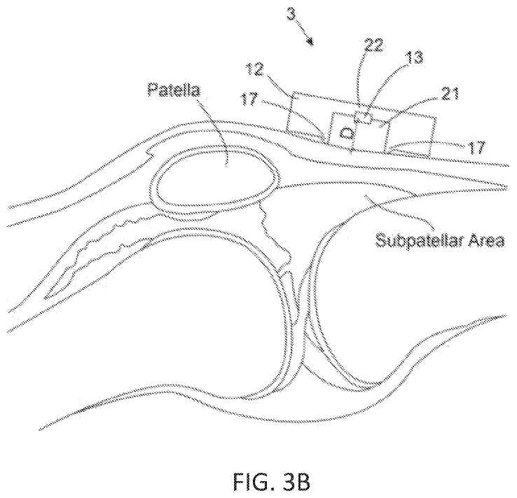

As shown in FIGS. 2 and 3, the main body of each frame 1, 2 can be rigid but elements or parts that will be in contact with skin of the subject or wearer can be made of a soft or flexible biocompatible material, for example elastic sensor frame 3 that is attached to first frame 1, for example with a screw, bolt, rived, snap-in connection, or other attachment mechanism, elastic sensor frame 3 including a soft layer 12 made of a biocompatible, elastic material, for example medical grade silicone that is “skin safe,” which can provide for a deformation and a movement of elastic sensor frame 3 relative to the skin, once positioned on the leg in vicinity of the knee joint. Moreover, surface 16 of elastic sensor frame 3 that comes into contact with skin can have anti-adhesive properties, for example by an anti-adhesion layer or surface treatment, to reduce a friction between skin and elastic sensor frame 3. This allows the surface 16 to smoothly move or slide relative to skin. In another variant, surface 16 of elastic sensor frame 3 can have adhesive properties, for example by an adhesion layer or surface treatment, for example to render the surface 16 softer or more elastic, or to provide for adherence, to increase friction or adherence between skin and elastic sensor frame 3. This allows the surface 16 to stick to the skin, avoiding any relative movement therebetween. It is also possible that both frames 1, 2 can communicate with each other by a data communication device 23, for example a first and second data communication device 23.1, 23.2 arranged respectively at the frames 1, 2. This can be a wireless communication device including but not limited to Bluetooth device, Infrared Data Association (IrDA) device, radio-frequency (RF) communication device, or other type of short distance communication interface, or a wired communication device such as but not limited to a Universal Serial Bus (USB) connection, I²C, CAN bus, other types of wired data bus or serial port connections.

The lower frame 1 can have two contact points or surfaces to the skin of the wearer, a first one embodied as a shin support portion 5 having a curved or concave shape to support lower frame 1 at a mid-section of the shin of a wearer, for example by removably attaching or securing shin support portion 5 with a strap 24 that can be placed around the calf muscle, and a second one being with the elastic sensor frame 3, elastic sensor frame 3 or a soft layer 12 thereof also having a curved or concave shape, configured to make contact area to the subpatellar or infrapatellar area of the knee joint, where the collection of acoustic emission is performed. Soft layer 12 or a portion of elastic sensor frame 3 can be made of a deformable biocompatible silicone material including a cavity 21 formed therein, to provide for an opening towards the skin of a user or wearer. The expressions subpatellar or infrapatellar are used interchangeably within the present patent specification.

Both shapes of the first and second contact points of first lower frame 1 with shin support portion 5 and portions of elastic sensor frame 3 are curved to fit the shape of the front surface of the shin ergonomically and allow a robust positioning of lower frame 1 in the anatomical coordinates. For example, it is possible that shin support portion 5, a part of acoustic sensor frame 3, or both, are custom made for each wearer, for example by a three-dimensional printing technique. A correct positioning of lower frame 1 along the proximal-distal axis can be performed by using the fibula head as a reference point to align shin support portion 5 of first lower frame 1, for example by having an inner surface shape that corresponds to a surface shape formed by the fibula head at the shin, for example a corresponding cavity that is matched with the fibula head protrusion or dent, and get the subpatellar contact point at the ideal position for acoustic sensor frame 3. The optional second upper frame 2 can have one contact point or surface to the skin to support the upper frame 2 for the arrangement at the distal part of the thigh of wearer, for example with thigh contact part 6 that has a concave or curved shape to fit ergonomically with a thigh of wearer, to be aligned with the anatomical coordinates, and can be removably attached or secured to thigh with a strap 25.

With this arrangement, it is possible to provide a minimal contact area and locations for first lower frame 1, to provide for an interconnection with the areas of the joint that minimize the creation of parasitic acoustic waves that could interfere with the acoustic signals generated by the knee, as compared to solutions where a knee brace is used. Also, it is possible to provide for robust, stable, yet flexible mechanical interconnection between acoustic sensor frame 3 and the skin of the knee, so that the knee and the skin thereof can still undergo movements whilst preserving a contact of acoustic sensor frame 3 with skin, to preserve a consistency of the acoustic measurements. For example, a flexibility and dimension of acoustic sensor frame 3 is such that a movement of patella during the motion of the knee does not or only insubstantially interferes with the placement of acoustic sensor frame 3, and therefore the airborne capturing of acoustic waves or sound.

As shown in FIG. 2, lower frame 1 can have a main body 26 that forms an enclosure for an electronic device, the electronic device for example a printed circuit board having a data processor device 18, a data communication device 23, and other electronic devices for the operation of smart wearable SW. An interconnection can be provided between electronic device located in main body 26 to electronic devices in elastic sensor frame 3, for example the acoustic sensor device 13, with cables or a flexible printed circuit board, and acoustic sensor frame 3 can be attached to main body 26 by a connection element 15, for example a bolt, protrusion, screws, or by a common casing element for both main body 26 and acoustic sensor frame 3, for example from a plastic mold casing having soft layer 12 attached thereto, to integrate at least partially both parts, to provide for a mechanically stable interconnection between these two elements.

FIG. 3A shows an exemplary elastic sensor frame 3 in more detail. Acoustic sensor frame 3 or at least a portion of elastic sensor frame 3 can be made of an elastic biocompatible material to form a skin contact surface that can be configured to be placed the subpatellar or infrapatellar area, for example a soft layer 12, soft layer 12, or sensor frame 3 having a cavity 21 formed therein, cavity 21 being open towards a skin attachment surface side of acoustic sensor frame 3. Acoustic sensor device 13 is arranged at cavity 21 such that an acoustic wave capturing element, for example a microphone membrane, charge plate, ribbon, diaphragm or over device that is configured to generate an electric signal from acoustic sound waves, is arranged inside the cavity to pick up airborne acoustic waves generated by the knee joint from an area of interest. In other words, cavity 21 and acoustic sensor device 13 are arranged such that upon placement of acoustic sensor frame 3 to subpatellar area of knee joint, is configured to capture acoustic waves that are propagated through the air inside cavity 21 from subpatellar area of knee joint. Therefore, acoustic sensor device 13 and cavity 21 are arranged such that at least sensing device or the acoustic wave capturing element is not in contact with the skin.

For example, acoustic sensor device 13 can include a first and a second microphone 13.1, 13.2, the first microphone 13.1 arranged to capture airborne medial subpatellar or infrapatellar acoustic emissions of the knee joint, the second microphone 13.2 arranged to capture airborne lateral subpatellar or infrapatellar acoustic emissions of the knee joint. Cavity 21 can be shaped to be perpendicular to an axis of extension of the tibia/fibula, and there can be two (2) cavities 21.1 and 21.2 for each respective microphone 13.1, 13.2, as exemplarily shown in FIG. 3A. Acoustic sensor device 13, for example first and second microphones 13.1, 13.2, can themselves be placed into a subcavity 22 that is located on the floor inside cavity 21, subcavity 22 shaped to accommodate acoustic sensor device 13. Acoustic sensor device 13 can be attached inside cavity 21 by being adhesively bonded to subcavity 22 by the use of a glue with elastic properties, for example a liquid silicon glue, or other type of glue that has a matching or similar elasticity in a dried state. The acoustic sensor device 13 can itself be packaged or wrapped into a casing, for example a casing that itself fits into subcavity 22, for example a casing having sound insulating properties to permit a reduction of artefact surrounding noise, and also for mechanical protection of acoustic sensor device 13.

Moreover, as exemplarily illustrated in the cross-sectional view of FIG. 3B, it is possible that a protrusion 17, for example a ridge, one or more knob, ledge, lip, bump or other type of distance providing device is arranged on the skin facing ergonomic surface 16 of acoustic sensor frame 3, substantially in close proximity to an edge where the cavity 21 is formed, with the goal to provide for a constant distance D between acoustic sensor device 13, and also to minimize a surface of contact between acoustic sensor frame 3 and the skin of knee, to avoid the creation of additional parasitic sound or acoustic waves. For example, protrusion 17 can be arranged as a bump next to cavity 21, or can be a ledge or lip that protrudes from the skin facing ergonomic surface 16 of acoustic sensor frame 3, and partially or fully surrounds cavity 21. In this respect, the one or more protrusions 17 can be configured to ascertain a substantially fixed and constant distance D from a skin surface of the subpatellar area of the knee of wearer during motion.

Acoustic sensor device 13, first and second microphones 13.1, 13.2, can be connected to main body 26 of lower frame 1 with by a flexible printed circuit or a cable that follows a guide 14 within the elastic material arranged at acoustic sensor frame 3. Main body 26 can enclose a substrate, for example a main printed circuit board, that is interconnected to flexible printed circuit board or cable that is in operative connection with first and second microphones 13.1, 13.2, for example via a connector, or by direct connection with the flexible and main printed circuit board. Main printed circuit board can be equipped with a data processor 18, for example a microcontroller to control signal acquisition, data processing, and to save of the data in memory. A kinematic sensor device 19, for example a first inertial measurement unit (IMU) or other device, can be arranged on main printed circuit board of main body 26, to measure and collect the kinematic information of a movement, position, and orientation of shin. A surface of extension of main printed circuit board can be arranged to be within main body 26 to be parallel with a direction of extension of the lower leg, and thereby can simplify a calibration process of the kinematic data. In addition, an electric power supply, for example a rechargeable battery or a solar panel, can be interconnected to main printed circuit board. Moreover, a top surface of main body 26 can include different user interface input and output devices, for example a switch button 4 to turn smart wearable SW on and off, and to initiate or start data acquisition, or to execute or start other functions of smart wearable SW.

Second upper frame 2 can also include a circuit board or other substrate to include different electronic components, for example a kinematic sensor device 20. For example, kinematic sensor 20 can be a second inertial measurement unit (IMU) or other position and motion measurement device, that can be arranged on circuit board of upper frame 2, or otherwise be attached or arranged to upper frame 2, to measure and collect the kinematic information of a movement, position, and orientation of the thigh. Furthermore, a user interface can be arranged on a top surface of upper frame 2 to provide for different user functions, for example a display screen 9 to provide for different information on status and operation of smart wearable SW, two or more push buttons 7, 8, that can be used by user to choose the exercise to perform, to start or stop the recording stand-alone, and other functions. Moreover, second upper frame 2 can be interconnected with a wired connection to lower frame, 1 for example for power supply and data communication by the use of a USB connection, but it is also possible that second upper frame 2 has its own power supply and is wirelessly connected with lower frame 1, for example with a wireless data interface for data communication, for example a Bluetooth interface.

As discussed above, the herein described embodiment proposes the use of acoustic sensors that capture airborne acoustic waves, instead of conventional contact microphones that would be in direct contact with the skin at the knee joint, by the use of an acoustic sensor frame 3 that is attached to a first frame 1. By doing so, subjective positioning of the sensors can be avoided, and thereby the of sensor loosening during acquisition is mitigated and prevented. In addition, the risks of patient infection are reduced. Moreover, to collect acoustic information from the knee joint in a robust manner, a mechanism is provided to be able to keep the sensors at a constant distance D from joint locations of interest during movement, for example the subpatellar or infrapatellar areas of the knee joint. One challenge is the movement of the skin and of the anterior ligament, which affects the shape of the knee. To address this issue, according to an aspect of the present invention, a solution is proposed where a deformable and elastic acoustic sensor frame 3 is used, for example having a soft layer 12, for example made of biocompatible silicone, and with the use of flexible printed circuits for operatively interconnecting the acoustic sensors 13. The sensors can be kept at a constant distance from the skin during movement by the help of protrusions 17, for example silicone protrusions that are in contact with the skin follows the knee movement. Furthermore, the silicone was chosen with an elasticity in a defined range, for example preferably in a Shore range between 50A and 80A, more preferably about 68A Shore, and fulfill medical device regulations for safety. Acoustic sensor 13 can collect data from both medial and lateral areas of the joints to improve the robustness of the data acquisition and to help to identify the location of any tissue damage in the joint: this is possible by synchronization of the data acquisition.

With respect to the operation of smart wearable SW, for example once first lower frame 1 with acoustic sensor frame 3, and optionally second upper frame 2 are attached to a knee joint of the wearer or subject, for example by using first and second attachment mechanism 24, 25, user can switch smart wearable SW for example with on/off switch 4. Next, as an example, push buttons 7, 8 of upper frame can be used to select different actions or programs, for example a movement regime that can be performed by the user to with the knee joint. As an example, the user or wearer could be instructed to perform a predefined knee motion routine, for example including ten (10) sit-to-stand movements, thereafter ten (10) flexion-extension movements, and thereafter walk for forty (40) meters. The start of the program could be signaled to the user with an acoustic signal with a speaker, vibratory alert, or a signal on the display screen 9. During the performance of the predefined knee motion routine, by the wearer, motion data from the kinematic sensor devices 19, 20, and data from acoustic sensor device 13, for example the first and second microphones 13.1, 13,2 can be recorded to the memory, time-stamped, and synchronized, controlled by data processor 18. Data processor 18 can also perform other data processing method and algorithms, including but not limited to filtering, median and average value generation, scaling and normalizing.

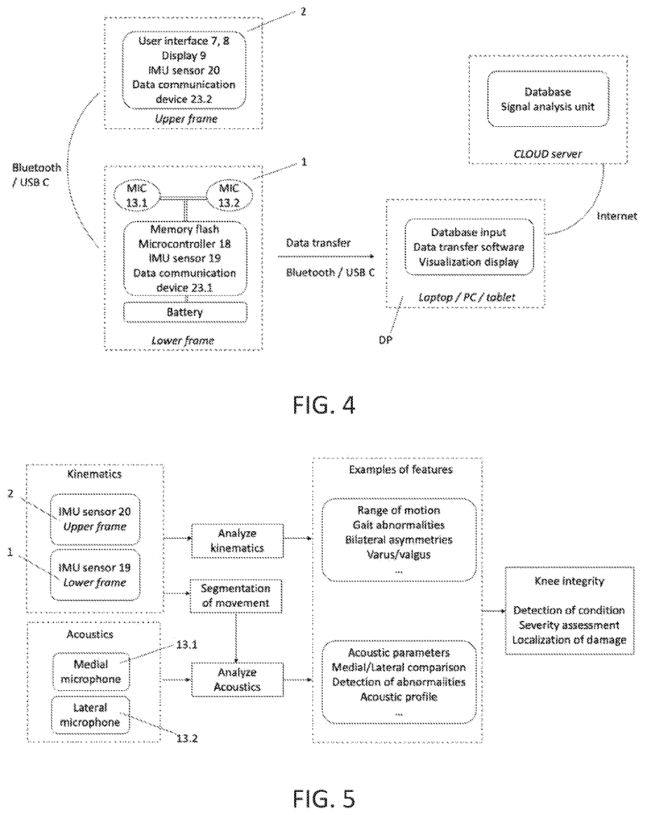

Thereafter, or even during the recording to smart wearable SW, user then just connect the first lower frame 1, second upper frame 2, or both, to a computer/laptop/tablet, for example data processing device DB, either by USB or by Bluetooth connection. A user interface can then transfer the data from the wearable locally, better encryption, and sending database, server, or cloud for automatic analysis. The results of the data analysis are then reported instantaneously to the user interface. An exemplary illustration of the system is shown in FIG. 4.

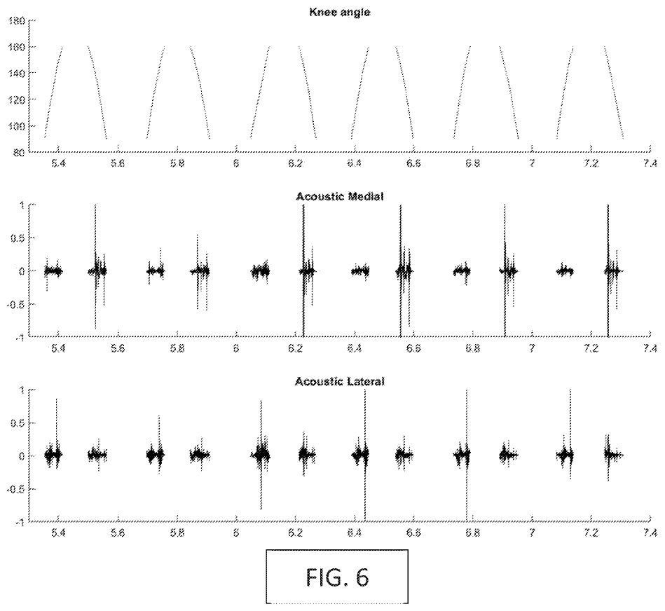

Also, as illustrated in FIG. 5, different data processing steps can be performed of a knee analysis software method as shown as a flowchart, for example with the use of data processing device DB. The data analysis can be divided into two parts: kinematic and acoustic. The kinematic data provides in itself parameters that provides information on the joint condition: for example, it is possible to quantify the full range of motion, the speed of specific movement, any bilateral asymmetries or abnormal shear movements characteristic of ligament damage or compartmental cartilage thinning. Furthermore, the kinematic information allows the segmentation of the acoustic data, as it is required to performed comparative analysis: for example, to evaluate knee crepitus at specific knee bending angles. Information from both medial and lateral compartment increases the robustness of the knee joint assessment, and also support the evaluation of compartmental damage which is relevant information for patient-targeted therapies. An example of segmented acoustic signals based on knee angles are presented in FIG. 6.

Also, it is possible that to smart wearables SW are attached to the left and the right leg, respectively, and these two smart wearables SW can be used simultaneously and synchronized for data analysis with data processing device DP. Furthermore, to replace the buttons/digital interface of the smart wearables and simplify the solution if two devices are used simultaneously, a version-controlled remotely by a tablet and Bluetooth can be used to start/stop the recordings of each exercise.

As indicated above, a smart wearable SW having solely the first lower frame 1 can be used, for example in a case where the range of motion of the knee angle is not necessary. For this option, it is possible that the recording can be controlled remotely by a wireless interconnection, for example Bluetooth, and the segmentation of the acoustic signals are done solely from the data collected by first kinematic sensor 19 of the lower frame 1.

With the herein described smart wearable SW, and a system including the smart wearable SW, and a method of operating the same, it is possible to perform an early stage analysis and detection of a knee joint for OA, in a non-intrusive and non-invasive fashion, which would allow to access a large panel of therapies and solutions to prevent/stop the disease progression. With the state of the art solutions, the time of the diagnosis is usually too late such that a knee replacement surgery may be the only remaining option, which is not only expensive, but also not fully satisfactory to the patients, as around 20% of knee replacement patients continue to experience joint pain even after a successful surgery. See Van Der Kraan et al., “Translation of Clinical Problems in Osteoarthritis into Pathophysiological Research Goals,” RMD open 2, No. 1, year 2016, p. e000224. Additionally, it is not a permanent solution for relatively young patients who require a more complicated revision surgery once the implant wears out. To summarize, the longer one can delay a knee replacement surgery, the better. In the last two decades, unfortunately, the rates of knee replacements have more than tripled. Finding a regimen to avoid a knee replacement surgery, which can cost around 30′000 USD in countries such as the United States, and managing OA patients appropriately in primary care can save substantial costs. See Hargraves, John, and Aaron Bloschichak. “International Comparisons of Health Care Prices from the 2017 IFHP Survey,” Blog. Health Care Cost Institute, Washington, DC (2019).

The progression of OA can be delayed or prevented if patients get on early-stage treatment regimens and adhere to them. However, to improve efficacy, the treatment solution should also be personalized to the patient. The clinical diagnostic information available today allows only very limited therapy customization. This challenge must be solved: to provide relevant biomarkers of the joint, representative of its function, for the physician to design a patient-specific solution. For example, the treating physician requires new specific metrics to recommend solutions such as custom knee braces or patient-specific rehabilitation programs focusing on a group of muscles to alleviate localized loads of the tissues. The physician also needs additional information to identify suitable patients for partial knee replacements or innovative pain-relief treatments (e.g., simulation-based with ultrasound, magnetic fields, electrotherapy). Emerging regenerative cartilage repair solutions based on tissue engineering, such as scaffolds and hydrogels, will also require new biomarkers not yet available clinically. Finally, the frequent monitoring of joint health is associated with high costs if performed with conventional modalities and practices. To enable a consistent periodic collection, follow-up and storage of the relevant biomarkers, the monitoring process needs to be fast, simple and automated with help from digital platforms.

In addition, while the examples described herein refer to the attachment of smart wearable SW to a knee joint, it is also possible that smart wearable SW be attached ton other types of joints for analysis, for example to

### REFERENCE NUMERALS

1 first, lower frame to be fixed to shin
2 second, upper frame to be fixed to thigh
3 flexible sensor frame
4 on/off switch
5 shin support portion for contact with skin
6 thigh contact part for contact with the skin
7 start/stop recording button
8. next test button
9 display device, LED screen
10 USB-C interface port
11 hole for straps
12 soft layer
13 acoustic sensors
14 guide for flexible printed circuit or for wire cable
15 protrusion or knob to connect to lower frame
16 ergonomic concave surface
17 protrusion or ridge around the microphone area
18 data processor device
19 first IMU sensor
20 second IMU sensor
21 acoustic sensor cavity
22 subcavity for accommodating acoustic sensor or casing for microphone
23 data communication device
24 first attachment mechanism, strap
25 second attachment mechanism, strap
26 main body

While the invention has been disclosed with reference to certain preferred embodiments, numerous modifications, alterations, and changes to the described embodiments are possible without departing from the sphere and scope of the invention, as defined in the appended claims and their equivalents thereof. Accordingly, it is intended that the invention not be limited to the described embodiments, but that it has the full scope defined by the language of the following claims.

1. A smart wearable for collecting data on an integrity of a joint of a wearer, the wearable comprising:
a first frame configured to be attached in proximity of the joint of the wearer, the frame including
an elastic sensor frame having an open cavity configured to be attached to an area of interest of the joint,
a kinematic sensor device configured to measure kinematics of the joint, and
acoustic sensor device arranged in the cavity configured to capture airborne acoustic emissions from the area of interest; and
a data processor device configured to receive and record data from the kinematic sensor device and from the acoustic sensor device.

2. The smart wearable according to claim 1, the joint being a knee joint, further comprising a second frame,
wherein the kinematic sensor device includes a first motion sensor and a second motion sensor,
wherein the first frame is attachable to an upper shin of the wearer, the first frame including the acoustic sensor device, and the second frame is attachable to a thigh of the wearer, and
wherein the first motion sensor is arranged at the first frame and the second motion sensor is arranged at the second frame.

3. The smart wearable according to claim 1, the joint being a knee joint,
wherein the cavity is configured to be placed at a subpatellar area of the knee joint, the acoustic sensor device arranged in the cavity to capture airborne acoustic emissions from the subpatellar area, a sensing area of the acoustic sensor device not contacting the skin of the wearer.

4. The smart wearable (SW) according to claim 3, wherein the acoustic sensor device includes a first and a second microphone, the first microphone arranged to capture airborne medial subpatellar acoustic emissions of the knee joint, the second microphone arranged to capture airborne lateral subpatellar acoustic emissions of the knee joint.

5. The smart wearable according to claim 1, wherein the kinematic sensor device includes an Inertial Measurement Unit (IMU).

6. The smart wearable according to claim 1, wherein the first frame includes a first attachment mechanism for attaching the first frame to the upper shin of the wearer.

7. The smart wearable according to claim 1, wherein the second frame includes a second attachment mechanism for attaching the second frame to the thigh of the wearer.

8. The smart wearable according to claim 1, further comprising:
a data communication device for transmitting data recorded from the kinematic sensor device and the acoustic sensor device to an exterior data processing device.

9. The smart wearable (SW) according to claim 2, wherein the first frame includes a first data communication device for transmitting data recorded from the first motion sensor and the acoustic sensor device, and the second frame includes a second data communication device for transmitting data recorded from the second motion sensor.

10. The smart wearable according to claim 1, wherein the elastic sensor frame is made of a biocompatible ergonomic material, or includes a soft layer having a biocompatible ergonomic pad fitting a shape of the upper shin.

11. The smart wearable according to claim 1, wherein the elastic sensor frame has a curved shape and includes a protrusion or ridge at an opening of the cavity.

12. The smart wearable (SW) according to claim 11, wherein the protrusion or ridge is configured to protrude away from surface and is arranged to at least partially surround the opening of the cavity.

13. The smart wearable according to claim 1, further comprising:
a user interface device, at least one element of the user interface device configured to receive a user instruction for starting a measurement by the smart wearable (SW).

14. A system for analyzing an integrity of a knee joint of a wearer, the system comprising:
a smart wearable (SW) for collecting data on the integrity of the knee joint according to claim 1, the smart wearable system including a data transmission device for transmitting data recorded from the kinematic sensor device and the acoustic sensor device; and
a data processing device configured to receive the data from the smart wearable (SW) and to analyze at least one of kinematics and acoustics of the knee joint.
